(19) United States
(12) Patent Application Publication
(10) Pub. No.: US 2007/0255190 A1
(43) Pub. Date: Nov. 1, 2007

(54) Exoskeleton System for a Proportional Movement Biological Segment and Exoskeleton Assembly of a Said Systems

(76) Inventor: Patrick Sadok, Lyon (FR)

Correspondence Address: CLARK & BRODY, 1090 VERMONT AVENUE, NW, SUITE 250, WASHINGTON, DC, 20005, US

(21) Appl. No.: 10/578,573

(22) PCT Filed: Nov. 5, 2004
(86) PCT No.: PCT/FR04/02850

§ 371 Date: Jan. 19, 2007

(30) Foreign Application Priority Data
Nov. 7, 2003 (FR) 03 13 087

(51) Int. Cl. A61F 5/00

(52) U.S. Cl. 602/16; 623/27; 602/24

(57) ABSTRACT

The subject of the invention concerns an exoskeletal system with: an exoskeletal weight-bearing structure composed of a reference structure and at least one mechanical segment, resources for acquiring movements and movement intentions, composed of resources for time related measurement of the effort coming from at least one biological segment and time-dependent resources for detecting the direction of the movements or movement intentions of these segments, resources for acquiring the spatial position of the mechanical segments in relation to the reference structure, operating resources providing the motor-power for the articulated mechanical segments, and control resources connected at their inputs to the movement and position acquisition resources, and at their outputs to the operating resources in order to control them.

This present invention concerns the technical area of assistance, in terms of support and motor-power, for biological segments, and in particular of the limb of a person, by means of a device called an exoskeleton.

Conventionally, a limb exoskeletal system, such as an orthosis, assists the biological limb of a user by partially or even completely relieving it of its own weight and of the efforts exerted by it. A limb exoskeleton is used to make up for a mobility deficiency of the limb or indeed to enhance its performance.

In previous designs, diverse exoskeleton implementation systems have been proposed. For example, U.S. Pat. No. 3,358,678 describes an exoskeletal device designed to be donned by the user, such as a garment. Such a device is controlled by pre-programmed sequences in order to keep the person in a stable erect position. In practice, it turns out to be difficult, or even impossible, for a handicapped person to fit such an exoskeletal structure, which is of a closed character. Moreover, such a device can be use only to keep a person stable so that such a device cannot be used to assist the limbs of the person in accordance with movement intentions.

Patent US 2003 11 59 54 describes an exoskeletal structure whose field of application is limited to tests and exercises designed for the upper limbs. The exoskeletal structure is equipped with a mechanical operating device of the counterweight type. Such a device has dimensions and mass which impose a stationary character on the assembly, thus explaining the limitation on the field of application. In addition, the use of a counterweight, which by definition applies forces of constant torque, does not allow the execution of natural movements.

Patent WO/95 32 842 describes an external appliance designed to be attached to a limb, on the segments to which it will apply torques. In its specification, such a device does not include a weight-bearing structure (on the chest or the pelvis for example) in relation to which the torques are applied to the limb, and it therefore cannot be applied to movements such as abduction of the arm, for example.

Patent JP 2002 346 960 describes a fixed and precise mechanical system with a specified number of segments and articulations, thus preventing adaptation to a particular pathology or application. The processor controlling the motor-power of this system takes no account of parameters applicable to the user and to the field of activities concerned, but uses as its sole input values that have been pre-defined and which vary according to angular positions and force signals. Such a system therefore presents variations of accuracy when controlling speed and force, since these parameters vary from one user to the next.

U.S. Pat. No. 3,449,769 describes an exoskeletal system with an exoskeletal weight-bearing structure equipped with resources for adaptation to the person, and composed of a reference structure supporting a series of mechanical segments connected together, and to the reference structure, by means of mechanical articulations. Such an exoskeletal system also includes sensors for acquiring the movements of the biological segments and sensors for acquiring the spatial position of the mechanical segments. Such sensors are connected as inputs to control resources which are connected at their outputs to on-off controlled fluid motors so as to generate the movement of the mechanical segments. It emerges that such an exoskeletal system cannot be used to reproduce the natural movements of the limbs and thus subjects the biological articulations to damaging stresses. Moreover, the movements of the exoskeletal system cannot be adapted to the pathology of the user or even to the movement intentions of the user.

This present invention therefore aims to remedy the drawbacks of previous designs by proposing an exoskeletal system that provides assistance in terms of support and motor-power for the biological segments of a person, where this assistance can be adapted optimally to the biomechanical and pathological characteristics of the person as well as to the movement intentions and the field of activities concerned.

In order to attain such an objective, the subject of the invention concerns an exoskeletal system with:
an exoskeletal weight-bearing structure equipped with resources for adaptation to the person, and composed of a reference structure and at least one mechanical segment connected to the reference structure by a mechanical articulation,
resources for acquiring the movements of the biological segments,
resources for acquiring the spatial position of the mechanical segments in relation to the reference structure,
operating resources providing the motor-power of the articulated mechanical segments,
and control resources connected at their inputs to the resources for acquiring movements and positions, and at their outputs to the operating resources in àrder to control them. According to the invention:
   the said movement acquisition resources also acquire the movement intentions, and are composed of resources for time-related measurement of the effort coming from at least one biological segment and time-dependent resources for detecting the direction of the movements or movement intentions of these segments,
   the said control resources include:
      control parameters applicable to the person and to the field of activities, and parameters applicable to the configuration of the exoskeleton,
      processing resources which, in accordance with the said parameters and information coming from the resources for acquiring movements or movement intentions, proportionately determine the characteristics of speed, acceleration, deceleration and effort of the said operating resources,
      and control resources used to control the said operating resources, according to characteristics of speed, acceleration, deceleration and effort that are determined beforehand by the said processing resources.

According to one advantageous characteristic, the control parameters applicable to the person and to the field of activities include the biomechanical and pathological characteristics of the person, in order to determine the proportionality factors of motor-power amplification, or attenuation where appropriate, and even removal of involuntary movements.

Advantageously, the control parameters include coefficients for limiting the amplitude of the person's movements.

In addition, it should be noted that U.S. Pat. No. 3,449,769 describes an exoskeletal structure whose different mechanical segments are articulated in relation to each other by simple pivot links whose axes are successively parallel or perpendicular. For reproduction of complex articular movement such as that of abduction of the human arm around the axis of the shoulder, it is planned to break these down into three successive pivot links whose two end axes are parallel to each other, and that in the middle is perpendicular to the other two. It should be noted however that an abduction of the arm by more than 130 degrees, while the arm can tolerate 180 degrees, causes the motor to collide with the head of the user. In the same sense, simplification of the articulation of the knee to a horizontal axis as recommended by U.S. Pat. No. 3,449,769 leads to undesirable stresses and friction due to the existence of the physiological valgus angle of the knee. Such an exoskeletal system, which includes articulation axes that do not correspond to the biological reality, cannot be used due to the efforts applied to the osseous segments, resulting in undesirable friction between the limb and the exoskeletal structure, and even to lesions.

In the same sense, U.S. Pat. No. 5,282,460 describes an exoskeletal system with an articulation having three axes that are mutually perpendicular and meeting in a point. Such an articulation exoskeletal undoubtedly results in stresses at the biological articulations of the user.

There is therefore a need to have an exoskeletal system whose exoskeletal structure can be adapted optimally to the biological segments and to the biological articulations of a person.

In order to attain such an objective, each mechanical articulation connecting two mechanical segments, or one mechanical segment in relation to the reference structure, includes:
resources for the adjustment of its position in relation to the reference structure or to another segment, in order to enable it to be positioned in relation to the biological articulation,
for each mechanical articulation corresponding to a biological articulation, with the exception of that of the shoulder, as many pivot links as the biological articulation has degrees of freedom,
for the mechanical articulation corresponding to the articulation of the shoulder, four degrees of freedom implemented by two pivot links and one pivot link sliding radially.

Advantageously, each pivot link is implemented by a shafted guidance system or by a shaftless guidance system.

Advantageously, each articulation of a mechanical segment is equipped, for each degree of freedom, with a biological articulation that has at least three degrees of freedom and at least one pivot link implemented by a shaftless guidance system, while the other pivot links are each implemented by a shafted guidance system.

Preferably, the shaftless guidance system is implemented by at least one circular rail section providing guidance for at least one mobile slide.

Advantageously, the radially-sliding pivot link is composed of several successive axes of rotation used to reproduce a trajectory that is close to that of the slide of the biological axis of rotation, or of a guide equipped with a template in which the axis of the pivot link describes a trajectory similar to this slide.

According to preferred examples of implementation, the resources for acquiring the movement or the movement intentions include:
stress gauges mounted in opposition on a fixed part connected to the weight-bearing structure, these being driven by a mobile part connected to a biological segment,
and/or resources for measuring the neuro-muscular stimuli sent by the person to his or her muscles.

Another purpose of the invention is to propose an exoskeletal weight-bearing structure that is capable of being fitted easily to a user while still bearing the different measuring sensors.

In order to attain such an objective, the resources for adaptation to the person are composed of a fixed part and a mobile part, which are concentric and each composed of two half-shells articulated axially to each other in order to allow the radial insertion of a biological segment.

According to a preferred implementation characteristic, each half-shell of the mobile part supports an adaptable membrane designed to be in contact with the biological segment and to be adapted to the morphology of the said biological segment.

Advantageously, the operating resources are composed of pneumatic muscles or linear pneumatic actuators.

Preferably, the weight-bearing structure includes adjustable end-stops for limiting the amplitude of movement of the articulated mechanical segments.

It should be noted that the control resources include programmed resources which are used to control the operation of the exoskeletal weight-bearing structure in accordance with specified sequences.

In addition, the control resources are preferably connected to input-output interfaces used to control and monitor, remotely in particular, the operation of the said exoskeletal system.

Advantageously, at least one mechanical segment, or indeed the reference structure, is fitted with mounting resources for additional structures.

The exoskeletal system of the invention includes a source of energy to power the control, acquisition and activation resources, carried by the exoskeletal weight-bearing structure and assuming a storable form, such as a battery or a fuel cell, or located close to the latter in order to supply it by means of a connection harness or by induction.

Advantageously, the exoskeletal weight-bearing structure provides assistance for a biological segment of a limb, the trunk or the pelvis of a person.

Another objective of the invention is to propose an exoskeletal assembly with several exoskeleton systems according to the invention and assembled by their reference structure onto a trunk and/or pelvic exoskeleton structure in order to constitute a partial or complete exoskeletal structure providing support and motor-power for miscellaneous biological segments of a person, either partially or completely.

Various other characteristics will emerge from the description provided below, with reference to the appended drawings which show, by way of non-limiting examples, different forms of implementation of the subject of the invention.

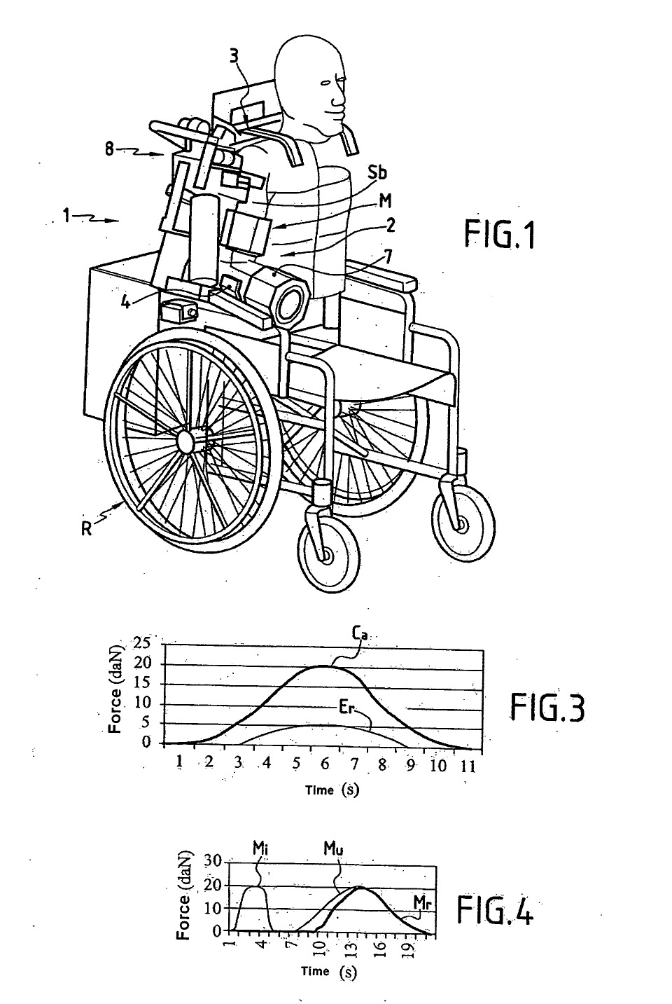

FIG. 1 is a view in perspective showing an example of implementation of an exoskeletal system for the upper right limb of a person seated in a wheelchair.

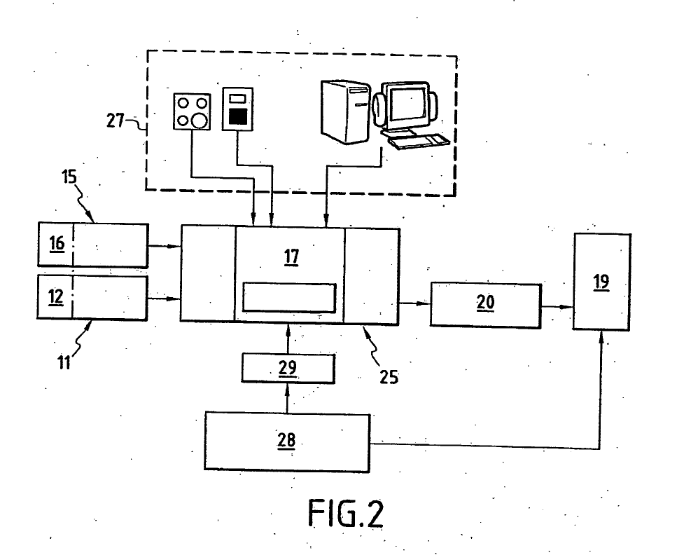

FIG. 2 is a functional block diagram of the control resources of the exoskeletal system of the invention.

FIGS. 3 and 4 represent time-related force curves illustrating certain characteristics of the exoskeletal system of the invention.

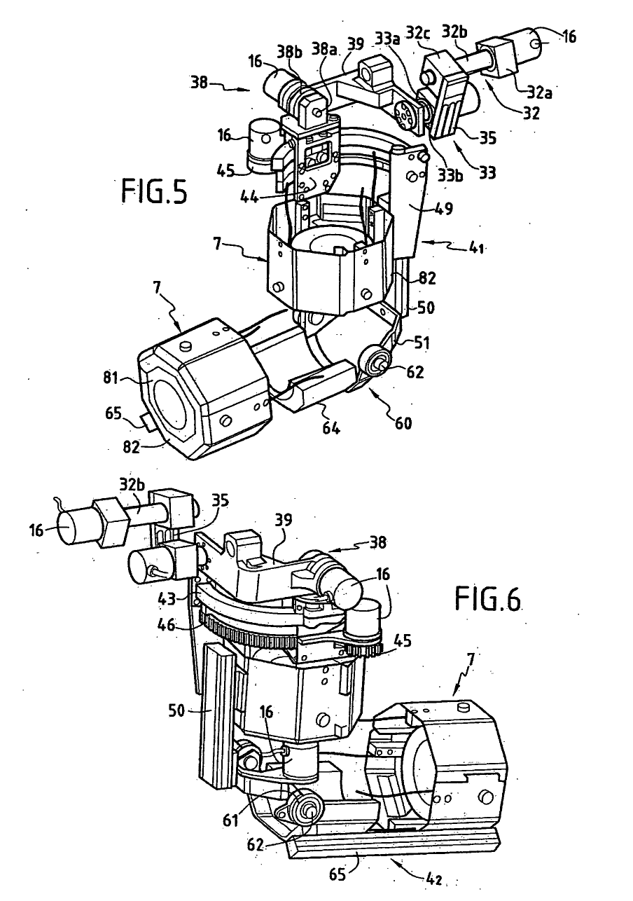

FIGS. 5 and 6 are simplified views in perspective of an exoskeletal system for the upper right limb of a person whose operating resources have been hidden in order to simplify the representation.

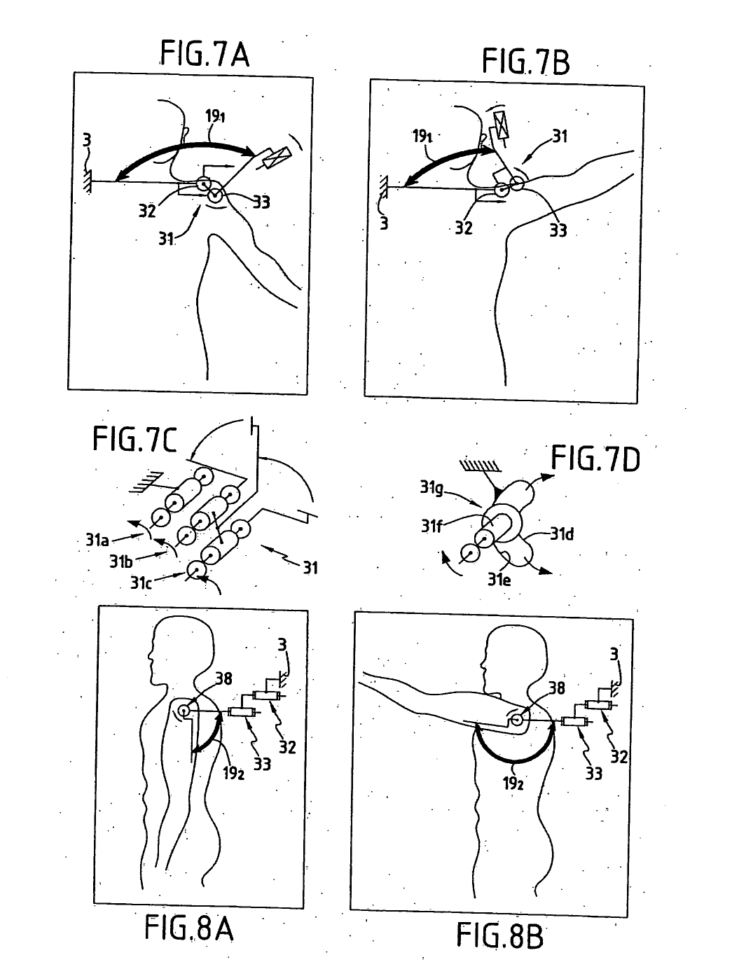

FIGS. 7A and 7B are kinetic diagrams illustrating the abduction movement of the arm.

FIGS. 7C and 7D are schematic views explaining some characteristics of the exoskeletal system of the invention.

FIGS. 8A and 8B are kinetic diagrams illustrating the flexing-extension movement of the arm.

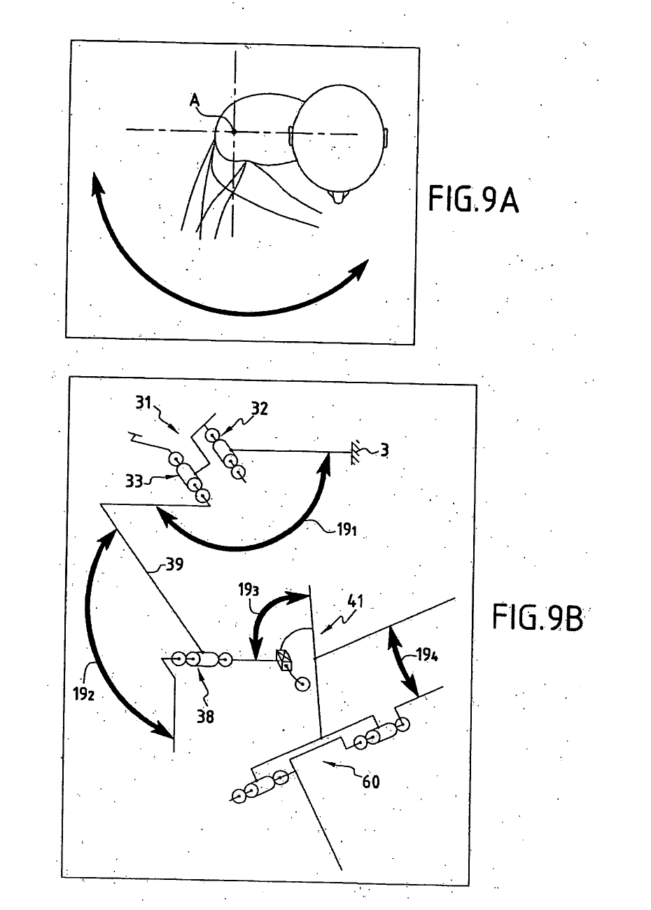

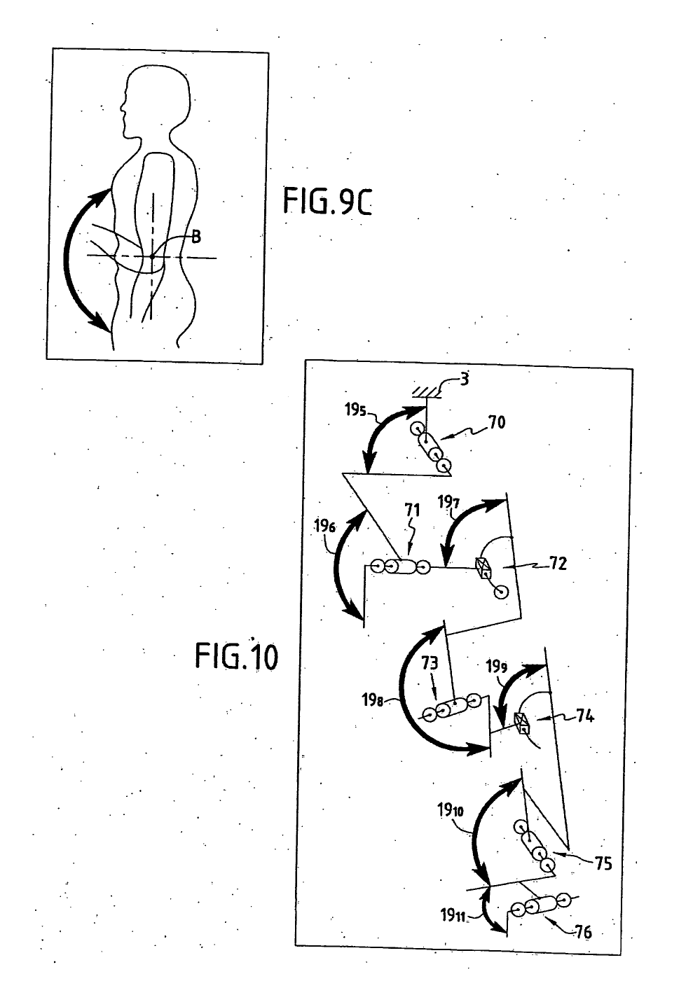

FIG. 9A is a kinetic diagram illustrating the rotation movement in relation to the longitudinal axis of the arm.

FIG. 9B is a schematic kinetic representation of the exoskeletal system illustrated in FIGS. 5 and 6.

FIG. 9C is a kinetic diagram illustrating the rotation movement of the forearm around the axis of the elbow.

FIG. 10 is a kinetic schematic representation of an exoskeletal system for the lower limb of a person.

FIG. 11 illustrates a preferred implementation variant in the open position of resources for adaptation of the exoskeletal system of the invention onto a biological limb.

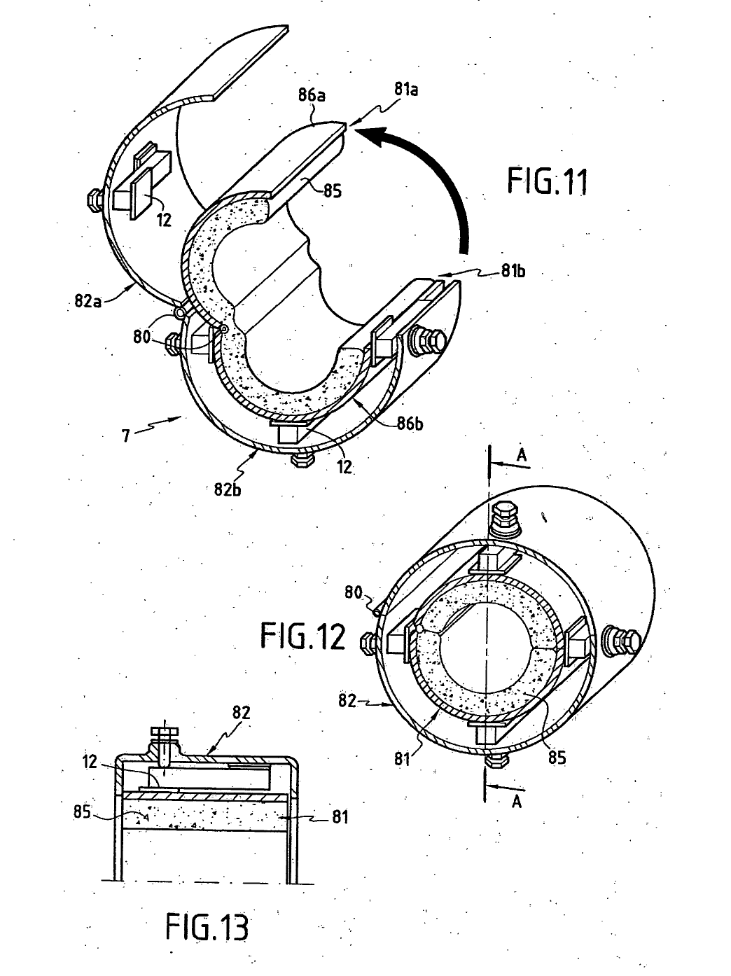

FIG. 12 illustrates a preferred implementation variant in the closed position of the resources for adaptation of the exoskeletal system of the invention shown in FIG. 11.

FIG. 13 is a view in partial longitudinal section taken more or less along lines AA of FIG. 12.

The subject of the invention concerns an exoskeletal system 1 designed to assist at least one biological segment S_{b} of one part M of a person in its movements by relieving it of all or part of the efforts exerted by it in order to execute tasks, or even to relieve it of its own weight or indeed to amplify its capabilities. It should be understood that the exoskeletal system 1 of the invention is designed to provide assistance, in a preferred manner, not only to one or more biological segments of the upper limb(s) such as the shoulder, the arm, the forearm or the wrist, but also one or more biological segments of the lower limbs such as the hip, the thigh, the leg or the foot. However, although the description that follows covers assistance to a biological segment of the limb of a person, the exoskeletal system 1 of the invention can be adapted to assist, as a part M of the person, the trunk or the pelvis of a person. In the example illustrated in FIG. 1, the exoskeletal system 1 provides assistance to the right arm and forearm of a person seated in a wheelchair R.

The exoskeletal system 1 of the invention includes an exoskeletal weight-bearing structure 2, composed of a reference structure 3 and at least one mechanical segment 4 designed to equip a biological segment S_{b} of the limb of a person. In the example illustrated, in which the exoskeletal system 1 is designed to provide assistance to the right shoulder, arm and forearm, the exoskeletal system 1 includes two mechanical segments 4. Each mechanical segment 4 is mounted on a corresponding biological segment S_{b}, using adaptation resources 7 of which a preferred implementation example will be illustrated in the remainder of the description. A mechanical articulation 8 is mounted between each adjacent mechanical segment 4 and between the reference structure 3 and the neighbouring mechanical segment 4.

It should be noted that the reference structure 3 is considered to be fixed in relation to the mechanical segment(s) whose purpose is to be mobile. This reference structure 3 can thus be supported either by the person, with the ability to move or not, or by a weight-bearing structure adjacent to the person, such as a wheelchair for example.

As illustrated more precisely in FIG. 2, the exoskeletal system 1 also includes resources 11 used to acquire the movements and the movement intentions of the biological segments S_{b}. These acquisition resources 11 are composed of resources providing firstly a time-related measurement of the effort coming from at least one biological segment S_{b}, and secondly a time-related detection of the direction of the movements or movement intentions of these biological segments.

According to a preferred implementation variant, these acquisition resources 11 include stress gauges 12 mounted in opposition on a fixed part connected to the weight-bearing structure 2 these being driven by a mobile part fixed onto a biological segment S_{b}. According to another implementation variant, the acquisition resources 11 include resources for measuring the neuro-muscular stimuli sent by the person to his or her muscles.

It should be understood that these acquisition resources 11 are used for time-related measurement of the effort exerted by the biological segment as well as its direction of movement, or in the event that the biological segment is not moved in space, by the movement intentions of the person.

The exoskeletal system 1 also includes resources 15 used to acquire the spatial position of the mechanical segments 4 in relation to the reference structure 3. These acquisition resources 15 can include angular position coders 16 for example.

The exoskeletal system 1 also includes control resources 17 connected at their inputs to the movement and position acquisition resources 11, 15 and at their outputs to operating resources 19 providing assistance in terms of support and motor-power to the mechanical segments 4.

The control resources 17 include control parameters applicable to the person and to the field of activities, as well as parameters applicable to the configuration of the exoskeleton.

The control parameters applicable to the person and to the field of activities concern in particular the measurements on each biological limb in order to determine their volume, allowing the control resources to determine the mass and then the inertia of each biological limb. Since the inertia has a tendency to generate a resistance opposing the movement, its value must be incorporated into the interpretation implemented by the processing resources 17.

These control parameters can possibly also concern the biomechanical characteristics of a person suffering from a motor handicap. Thus, as illustrated in FIG. 3, in the case of a limb that is suffering from a permanent or temporary lack of mobility potential, it is possible to effect a precise measurement of its residual effort potential Eᵣ in order to compensate for the latter by restoring biomechanical capabilities to it that are greater than its own. Thus, it is possible to achieve an enhanced capacity Cₐ which corresponds to an amplification of motor-power.

Likewise, in the case of re-educating the limb of a person, it is possible to envisage defining a re-education parameter. In this case, the person suffers from a temporary restriction of motor-power. It is equally possible to envisage a progressive reduction of the power assistance to the exoskeletal system and/or a progressive increase in the work-rate of the exoskeletal system according to firstly the time and secondly the increase, as re-education of the residual capabilities of the person progresses.

FIG. 4 illustrates another example of a control parameter concerning the case of a person suffering from involuntary movements commonly referred to as “overboost”. The movements of the person are quantified in a first stage in order to separate the voluntary movement Mu from the involuntary movement Mi. Then the movement restored Mr by the exoskeletal system 1 is used to attenuate or even to remove the involuntary movement Mi.

The control parameters applicable to the field of activities of the person can be fixed load parameters in the event that the exoskeletal system receives additional structures such as heavy protective elements. The exoskeletal system of the invention then acts as a load shedder, relieving the person of this encumbering mass. The mass and the centre of gravity of each of these additional elements are measured and configured in the form of fixed load parameters. Such additional structures can be composed of ball-protection, fire-protection or anti-crush clothing for example.

The control parameters applicable to the field of activities of the person can also be composed of adjustable load parameters in the event that the person uses appliances, tools, arms, or miscellaneous accessories requiring very precise movements, very large efforts or indeed the handling of a heavy load. In this case, the exoskeletal system provides an increase of the capabilities of the person whose coefficient of multiplication will be dimensioned in relation to the needs of the person and the field of activities concerned.

The adjustable load parameters can also be composed of acceleration or deceleration factors, or of large values in the event that the exoskeletal system performs the role of an anti-G suit for example. The control resources 17 receive information in real time, connected with these accelerations and/or decelerations, with a view to converting it into a force multiplication coefficient aimed at opposing the inertial factor.

It should be noted that the control parameters applicable to the person include coefficients to limit the spatial amplitude of the person's movements.

The parameters applicable to the configuration of the exoskeleton are composed of the characteristics of the various components of the exoskeletal weight-bearing structure 2, such as the dimensions, masses, and centres of gravity, as well as the characteristics of the acquisition resources 11, 15, the power of the operating resources 19 and the energy used.

According to another characteristic of the invention, the control resources 17 include processing resources which proportionately determine characteristics of speed, acceleration, deceleration and effort for the operating resources 19, in accordance with the control parameters applicable to the person and to the field of activities, the parameters applicable to the configuration of the exoskeleton, and the information on the movements or movement intentions, coming from the acquisition resources 11.

These characteristics of speed, acceleration, deceleration and effort determined by these processing resources are used by control resources 20 to control the operating resources 19 according to such characteristics of speed, acceleration, deceleration and effort.

It emerges from the foregoing that the exoskeletal system 1 of the invention implements a proportionality relationship between the movement emitted or intended by the person and that reproduced by the exoskeletal weight-bearing structure. Thus the movement of the exoskeletal weight-bearing structure is a function of the signals relating to the movements or movement intentions of the person, the control parameters applicable to the person and to the field of activities, and the parameters applicable to the configuration of the exoskeleton.

Thus, this proportionality determines a movement generated by the operating resources 19 and transmitted to the exoskeletal weight-bearing structure 2, whose speed, acceleration, deceleration and effort characteristics are a function of the input data received by the control resources 17. These input data are thus corrected by the various parameters described above.

It should be noted that the control resources 17 can form part of a control device 25 connected at their inputs to the measuring sensors 12, 16 and at their outputs to the control resources 19. It is clear that such a control device 25 can include the processing resources of the signals delivered by the sensors 12, 16 which, in this hypothesis, are connected as inputs to the control device 25.

Such a control device 25 is fitted with input and output interfaces 27 used to control and monitor the operation of the exoskeletal system. These input and output interfaces 27 can be located remotely or in the environment close to the exoskeletal system, being carried, for example, by the reference structure 3 or by a support adjacent to the person, such as a wheelchair. These input and output interfaces 27 can, for example, take the form of a control unit, a man/machine interface or a computer connected by a line link or not.

It should be noted that the control resources 17 include programmed resources used to control the operation of the exoskeletal weight-bearing structure 2 in accordance with specified sequences. Such sequences can be triggered by the input and output interfaces 27.

The exoskeletal system 1 also includes a source of energy 28 designed to power the various elements constituting the exoskeletal system, such as the acquisition resources 11, 15, the operating resources 19 and the control resources 17, preferably through a protection circuit 29. This energy source can be in storable form, such as a battery or a fuel cell carried by the exoskeletal weight-bearing structure and in particular by the reference structure 3. This energy source can also be located close to the exoskeletal system and powers the various component elements by means of a connection harness or by induction.

According to another aspect of the invention, the exoskeletal system 1 includes an exoskeletal weight-bearing structure which is used to conform to the bio-mechanical movements of each biological segment of the person. Thus, each mechanical articulation 8 connecting together two mechanical segments 4 or a mechanical segment 4 in relation to the reference structure 3, includes resources for the adjustment of its position in relation to the reference structure 3 or another mechanical segment 4, in order to enable it to be positioned in relation to the biological articulation. It is intended that adjusting the position of a mechanical articulation 8 firstly involves adjustment of the distance that separates it from the neighbouring articulation and secondly adjustment of the inclination of each of the axes that constitute this mechanical articulation.

In addition, each mechanical articulation 4 corresponding to a biological articulation, with the exception of that of the shoulder, includes as many pivot links as the biological articulation has degrees of freedom. In other words, other than the shoulder, each degree of freedom of a biological articulation is implemented by a pivot link which, by definition, has one degree of freedom in rotation. For its part, the mechanical articulation corresponding to the articulation of the shoulder includes four degrees of freedom implemented by two pivot links and a radially-sliding pivot link, corresponding to three degrees of freedom in rotation and one degree of freedom in translation.

Each pivot link is implemented by a shafted guidance system or by a shaftless guidance system. Note that a pivot link can be composed of several partial and coaxial pivot links. In the case of a biological articulation that does not have more than two degrees of freedom, such as the elbow, the wrist or the knee, each degree of freedom of the corresponding mechanical articulation is implemented by a pivot link composed of a shafted or shaftless guidance system. In the case where the biological articulation includes at least three degrees of freedom (the shoulder, the hip or the foot), at least one of the three pivot links is implemented by a shaftless guidance system, while the other pivot links are each implemented by a shafted guidance system.

According to one advantageous aspect of the invention, the simple or sliding pivot links are positioned in a hierarchical movement tree-structure in which each is supported by the mechanical articulation that precedes it. Thus, for the upper limb, the exoskeletal system 1 of the invention takes the form of a hierarchical succession of the following movements—abduction of the shoulder, flexing-extension of the shoulder, longitudinal rotation of the arm around its axis, flexing-extension of the elbow, longitudinal rotation of the forearm around its axis, flexing-extension of the wrist, and abduction/adduction of the wrist. Likewise, for the lower limb, the exoskeletal system 1 of the invention takes the form of a hierarchical succession of the following movements—abduction of the hip, flexing-extension of the hip, rotation of the leg around its longitudinal axis at the level of the hip, flexing-extension of the knee, rotation of the leg around its longitudinal axis at the level of the knee, abduction/adduction of the foot, and flexing-extension of the foot.

FIGS. 5 and 6 show an example of implementation of an exoskeletal system to assist the right shoulder, arm and forearm of a person, so that the last two movements associated with the wrist are not assisted in the exoskeletal system illustrated in the drawings.

Rotation of the human shoulder corresponding to the abduction movement of the arm (FIGS. 7A and 7B) is a rotation movement combined with a sliding of its instantaneous rotation centre. Biomechanically speaking, an acceptable simplification can be the combination of a rotation (from 0 to 90 degrees) and then a simultaneous sliding and rotation (from 90 to 180 degrees). According to the invention, this movement of the articulation of the shoulder is implemented by a radially-sliding pivot link 31. According to an implementation example illustrated in FIG. 7C, this radially-sliding pivot link 31 can take the form of several axes of rotation of simple pivot links 31a, 31b, 31c mounted successively in series, and used to reproduce a trajectory close to that of the sliding of the biological axis of rotation. It should be noted that this radially-sliding pivot link 31 can be implemented in a different way, as illustrated in FIG. 7D for example, by means of a guide 31d equipped with a template 31e in which an axis 31f of rotation of the pivot link 31g can describe a radial trajectory similar to the desired sliding action.

In the implementation example illustrated in FIGS. 5 and 6, and as explained above, the radially-sliding pivot link 31 takes the form of two pivot links 32, 33, each implemented by a shafted guidance system. In general, each shafted pivot link can be implemented for example, either by assemblies of the shaft-plus-housing type, or by shaft-plus-housing assemblies equipped with bearing fittings of all types, or by shaft-housing assemblies equipped with rings that include materials with a low coefficient of friction, or again by bearings using a high-pressure fluid arrangement such as a hydraulic bearing.

The first pivot link 32 thus has a housing 32a connected to the reference structure 3 and a rotating shaft 32b whose angular position in relation to this reference structure 3 is detected by a coder 16. The second pivot link 33 is implemented by a shaft 33a mounted in a housing 33b which is carried by a plate 35 to which is also fixed the enclosed end 32c of the rotating shaft 32b. The housing 33b is mounted in an adjustable manner on the plate 35 so as to allow adjustment of the relative spacing between the rotating shafts 32b, 33a. A coder 16 is positioned to detect the angular position of the rotating shaft 33a in relation to this plate 35.

An activation resource 19₁ generates the abduction movement, which takes place in two stages. The first rotation is effected around the second pivot link 33 from 0 to 90 degrees until the end of the activation resource 19₁ comes up against a mechanical end-stop at the enclosed end 32c. The second rotation then takes place around the first pivot link 32 on a trajectory of 90 to 160 degrees.

The exoskeletal system 1 then aims to reproduce the flexing-extension movement of the shoulder as illustrated more precisely in FIGS. 8A and 8B. Such a degree of freedom of the biological articulation takes the form of a pivot link 38 implemented by a shafted guidance system. This pivot link 38 includes a rotating shaft 38a mounted in a housing 38b which is connected to the rotating shaft 33a of the second pivot link 33 by means of a bracket 39. The shaft 38a is equipped with a position coder 16. The housing 38b includes resources for the adjustment of its position in relation to the other pivot links. An activation resource 19₂ acting on the bracket 39 is used to provide motor drive for the flexing-extension movement of the shoulder.

FIGS. 9A and 9B illustrate the third degree of freedom of the shoulder articulation, namely the longitudinal rotation of the arm around its longitudinal axis A. This degree of freedom is implemented by means of a pivot link 41 in the form of a shaftless guidance system. As mentioned above, at least one shaftless guidance system is required for an articulation with at least three degrees of freedom, due to the space required to implement the different pivot links.

The shaftless guidance system 41 is composed of at least one circular rail section 43 centred on an axis comprising longitudinal rotation axis A of the arm around its axis. This rail section 43 provides guidance in rotation around axis A of at least one mobile slide 44 supporting the housing 38b of the shaft belonging to the third pivot link 38. The mobile slide 44 is angularly adjustable in relation to the pivot link 38. Such a slide 44 is fitted with sliding interfaces in materials with a low coefficient of friction, or bearing elements such as balls, rollers, needles or ball-bearings. The slide 44 is fitted with a coder 16 used to ascertain its position around axis A. For example, this coder 16 includes a pinion 45 engaging with a rack 46 carried by the circular guide rail 43. The longitudinal rotation movement of the arm around its longitudinal axis A is provided by operating resources shown as 193.

This guidance rail 43 is carried by a mechanical segment 41 that takes the form of a vertical stringer 49 carrying a slide 50 on which is mounted a yoke 51 carrying the mechanical articulation of the elbow. The ability to adjust the run of the slide 50 in relation to the vertical stringer 49 allows adjustment of the distance between the articulation of the shoulder and the articulation of the elbow.

As shown more precisely in FIG. 9C, the exoskeletal system then aims to implement the rotation of the forearm around the axis B of the elbow by means of a pivot link 60. This pivot link 60 is implemented by a shafted guidance system with, in the example illustrated, two housings 61 carried by the ends of the yoke 51 and in which are housed two half-shafts 62 mounted coaxially with each other and associated with a support half-shell 64 for the forearm forming part of a mechanical segment 4₂. The rotation movement of the elbow around the axis 62 is provided by operating resources 19₄. A coder 16 is used to determine the rotation position of the axis 62. The half-shell 64 supports a lower stringer 65 which is mounted on the half-shell 64 preferably in an adjustable manner. It should be noted that in this case, the pivot link 60 takes the form of two partial coaxial pivot links.

It emerges from the foregoing description that the exoskeletal system 1 of the invention is used to conform optimally to the bio-mechanical movements of each biological segment of the person, by the positioning, for each biological articulation, of a mechanical articulation 8 connected to the reference structure 3, or to another mechanical articulation 8, by means of a mechanical segment 4. In the implementation example described in FIGS. 5 and 6, the exoskeletal system 1 includes, as a mechanical articulation 8, radially-sliding pivot link 31, pivot link 38, pivot link 41 and pivot link 60. Likewise, the exoskeletal system 1 includes, as a mechanical segment 4, from the reference structure 3, bracket 39, mechanical segment 41 (composed of vertical stringer 49, slide 50 and yoke 51), mechanical segment 42 composed of support half-shell 64, and lower stringer 65.

The operating resources 19, 19₁, 19₂, etc. are preferably of the pneumatic type. These operating resources can be composed of double or single effect linear actuators or double effect rotating actuators. According to a preferred implementation variant, the operating resources are implemented by single-effect actuators commonly called pneumatic muscles, like those marketed by the Festo company under the “Mas” references. According to this implementation example, which includes operating resources of the pneumatic type, the energy source 28 powers a pneumatic compressor which itself powers the control resources 20. These control resources 20 in turn provide the pneumatic operating resources with proportional work-rate and proportional pressure. This pneumatic compressor can be carried by the exoskeletal weight-bearing structure 2 or be located nearby, being connected to the control resources 20 by a power-supply harness.

The exoskeletal system 1 described above aims to provide assistance to the first two biological segments of the upper limb of a person. Of course the exoskeletal system of the invention can be adapted to provide assistance to a segment, and more generally to other biological segments, of the lower limb of a person. According to this implementation variant, the exoskeletal system of the invention takes the form of a hierarchical succession of the following movements: abduction of the hip, flexing-extension of the hip, rotation of the leg around its longitudinal axis at the level of the hip, flexing-extension of the knee, rotation of the leg around its longitudinal axis at the level of the knee, abduction/adduction of the foot, and flexing-extension of the foot. Thus as illustrated more precisely in FIG. 10, the exoskeletal weight-bearing structure 2 includes, successively from the reference structure 3:
a pivot link 70 implemented by a shafted guidance system and creating the degree of freedom corresponding to abduction of the hip,
a pivot link 71 implemented by a shafted guidance system and creating the degree of freedom corresponding to flexing-extension of the hip,
a pivot link 72 implemented by a shaftless guidance system and corresponding to the degree of freedom of rotation of the leg around its longitudinal axis at the level of the hip,
a pivot link 73 implemented by a shafted guidance system and corresponding to the degree of freedom of flexing-extension of the knee,
a pivot link 74 implemented by a shaftless guidance system and corresponding to the degree of freedom of rotation of the leg around its longitudinal axis at the level of the knee,
a pivot link 75 implemented by a shafted guidance system and corresponding to the degree of freedom of abduction/adduction of the foot,
a pivot link 76 implemented by a shafted guidance system and corresponding to the degree of freedom of flexing-extension of the foot.

The movements of abduction of the hip, flexing-extension of the hip, rotation of the leg around its longitudinal axis at the level of the hip, flexing-extension of the knee, rotation of the leg around its longitudinal axis at the level of the knee, abduction/adduction of the foot and flexing-extension of the foot, are all provided by operating resources 19₅ to 19₁₁ respectively.

As explained in the foregoing description, the exoskeletal weight-bearing structure 1 is equipped with resources 7 for adaptation to the biological segment of the limb to be assisted.

According to another aspect of the subject of the invention, the exoskeletal system 1 is fitted with adaptation resources 7 that are designed to allow more reliable and easier installation of the biological segment(s) of the person while also providing effective acquisition of the movements or movement intentions of the biological segments of the person.

As illustrated more precisely in FIGS. 11 to 13, these adaptation resources 7 take the form of a bracelet or cuff that opens along an axis 80 lying in a direction that is more or less parallel to the axis of the biological segment in order to enable easy fitting and removal of the corresponding biological segment. Such a bracelet 7 includes a mobile part 81 connected to a biological segment S_{b} and a fixed or reference part 82 connected by any appropriate means to the weight-bearing structure and more precisely to a mechanical segment 4. Thus, in the example illustrated in FIGS. 5 and 6, the fixed part 82 of each bracelet 7 is fixed onto a slide (50 and 65 respectively).

The fixed part 82 and the mobile part 81 are more or less concentric and are each composed of two half-shells, (82a-82b and 81a-81b, respectively) articulated axially with each other along an axis 80. Each half-shell 81a-81b of the mobile part supports an adaptable membrane 85, which can be inflatable for example, designed to be in contact with the biological segment and to be adapted to the morphology of the biological segment. The adaptable membrane 85 thus encloses a biological segment when the bracelet 7 is closed. It should be considered that the adaptable membrane best encloses the biological segment either when bare or covered with a garment.

Each fixed part 82 is equipped with stress gauges 12 mounted in opposition. In the example illustrated, the fixed part 82 is equipped with four stress gauges 12 which are angularly offset by 90 degrees so as to form two pairs in opposition. The stress gauges 12 are designed to make contact with a support plate 86a-86b forming part of the two mobile half-shells and supporting the adaptable membrane 85.

The bracelet 7 described above thus includes an adaptable membrane 85 which is a sort of mobile internal bracelet that receives the start of movements generated by the limb. Such an internal bracelet is used to activate the stress gauges 12, which deform in proportion to the pressure exerted by the mobile part.

It should be noted that the bracelet 7 can be provided with an additional degree of freedom in order to allow rotation, around the longitudinal axis, between the internal bracelet 81 and the fixed part 82.

The subject of the invention has been described mostly for an exoskeletal system assisting a biological limb. It is clear however that the same exoskeletal structure 2 can be used to provide assistance, support and motor-drive for the trunk and/or the pelvis of a person. This exoskeletal structure is articulated in the same way as the limb exoskeleton, by pivot links that coincide with the degrees of freedom of this assembly. Onto this exoskeletal structure, whose pelvis is similar to the fixed structure and the trunk to a biological segment or vice-versa, one or more exoskeleton limb systems according to the invention can be assembled by their reference structures 3 to form an exoskeletal assembly suitable for the different limbs of a person. This assembly can then constitute a complete or partial exoskeletal structure, so as to provide support and motor-power to various biological segments of a person, in a complete or partial manner.

The invention is not limited to the examples described and illustrated, since various modifications can be made to it without moving outside of its scope.

1. An exoskeletal system providing assistance in terms of support and motor-power for at least one biological segment (S_{b}) of a person, where this system has:
an exoskeletal weight-bearing structure equipped with resources for adaptation onto the person, and composed of a reference structure and at least one mechanical segment connected to the reference structure by a mechanical articulation.
resources for acquiring the movements of the biological segments,
resources for acquiring the spatial position of the mechanical segments in relation to the reference structure,
operating resources providing motor-power to the articulated mechanical segments,
and control resources connected at their inputs to the movement and position acquisition resources, and at their outputs to the operating resources, in order to control them, characterised in that:
the said resources for acquiring the movements also acquire the movement intentions, and are composed of resources for time related measurement of the effort coming from at least one biological segment and time-dependent resources for detecting the direction of the movements or movement intentions of these segments,
the said control resources include:
control parameters applicable to the person and to the field of activities, and parameters applicable to the configuration of the exoskeleton,
processing resources which, according to the said parameters and the information coming from the resources for acquiring movements or movement intentions, proportionately determine characteristics relating to speed, acceleration, deceleration and effort for the said operating resources,
and control resources used to control the said operating resources, in accordance with characteristics of speed, acceleration, deceleration and effort determined beforehand by the said processing resources.

2. An exoskeletal system according to claim 1, characterised in that the control parameters applicable to the person and to the field of activities include the biomechanical and pathological characteristics of the person, in order to determine the proportionality factors of motor-power amplification, and attenuation where appropriate, or even removal of the involuntary movements.

3. An exoskeletal system according to claim 1, characterised in that the control parameters include coefficients for limiting the amplitude of the person's movements.

4. An exoskeletal system according to claim 1, characterised in that each mechanical articulation connecting two mechanical segments or one mechanical segment in relation to the reference structure includes:
resources for the adjustment of its position in relation to the reference structure or another segment, in order to enable it to be positioned in relation to the biological articulation,
for each mechanical articulation corresponding to a biological articulation, with the exception of that of the shoulder, as many pivot links as the biological articulation has degrees of freedom,
for the mechanical articulation corresponding to the articulation of the shoulder, four degrees of freedom implemented by two pivot links and a radially-sliding pivot link.

5. An exoskeletal system according to claim 4, characterised in that each pivot link is implemented by a shafted guidance system or by a shaftless guidance system.

6. An exoskeletal system according to claim 4, characterised in that each articulation of a mechanical segment is equipped, for each degree of freedom of a biological articulation, with at least three degrees of freedom, and at least one pivot link implemented by a shaftless guidance system, while the other pivot links are each implemented by a shafted guidance system.

7. An exoskeletal system according to claim 6, characterised in that a shaftless guidance system is implemented by at least one circular rail section providing guidance for at least one mobile slide.

8. An exoskeletal system according to claim 4, characterised in that the radially-sliding pivot link is composed of several successive axes of rotation used to reproduce a trajectory close to that of the slide of the biological axis of rotation or of a guide equipped with a template in which the axis of the pivot link describes a trajectory similar to this slide.

9. An exoskeletal system according to claim 1, characterised in that the resources for acquiring the movement or the movement intentions include:
stress gauges mounted in opposition on a fixed part connected to the weight-bearing structure, these being driven by a mobile part connected to a biological segment,
and/or resources for measuring the neuro-muscular stimuli sent by the person to his or her muscles.

10. An exoskeletal system according to claim 9, characterised in that the fixed part and the mobile part are concentric and each composed of two half-shells articulated axially to each other to allow the radial insertion of a biological segment.

11. An exoskeletal system according to claim 10, characterised in that each half-shell of the mobile part supports an adaptable membrane designed to be in contact with the biological segment and to be adapted to the morphology of the said biological segment.

12. An exoskeletal system according to claim 1, characterised in that the operating resources are composed of pneumatic muscles or linear pneumatic actuators.

13. An exoskeletal system according to claim 1, characterised in that the weight-bearing structure includes adjustable end-stops for limiting the amplitude of movement of the articulated mechanical segments.

14. An exoskeletal system according to claim 1, characterised in that the control resources include programmed resources used to control the operation of the exoskeletal weight-bearing structure in accordance with specified sequences.

15. An exoskeletal system according to claim 1, characterised in that the control resources are connected to input-output interfaces used to control and oversee, remotely in particular, the operation of the said exoskeletal system.

16. An exoskeletal system according to claim 1, characterised in that at least one mechanical segment or indeed the reference structure is fitted with mounting resources for additional structures.

17. An exoskeletal system according to one of claims claim 1, characterised in that it includes an energy source feeding the control, acquisition and activation resources, carried by the exoskeletal weight-bearing structure and assuming a storable form such as a battery or a fuel cell, or located close to the latter in order to supply it by means of a connection harness or by induction.

18. An exoskeletal system according to claim 1, characterised in that the exoskeletal weight-bearing structure (2) provides assistance to a biological segment of a limb, or the trunk or pelvis of a person.

19. An exoskeletal assembly with several exoskeleton systems for biological segments, according to claim 1, and assembled by their reference structure onto an exoskeleton structure for the trunk and/or the pelvis, in order to constitute a partial or complete exoskeletal structure support and motor-power for miscellaneous biological segments of a person, either partially of completely.

20. An exoskeletal system according to claim 5, characterised in that each articulation of a mechanical segment is equipped, for each degree of freedom of a biological articulation, with at least three degrees of freedom, and at least one pivot link implemented by a shaftless guidance system, while the other pivot links are each implemented by a shafted guidance system.
